(19) United States
(12) Patent Application Publication
(10) Pub. No.: US 2025/0186231 A1
(43) Pub. Date: Jun. 12, 2025

(54) DELIVERY SYSTEM FOR GASTRO-INTESTINAL IMPLANT

(71) Applicant: BariaTek Medical, Paris (FR)

(72) Inventors: Yonatan GRAY, Paris (FR); Youssef BIADILLAH, Maisons-Laffitte (FR); Christophe NAZ, Fontainebleau (FR); Anthony LE BRETON, Paris (FR)

(73) Assignee: BariaTek Medical, Paris (FR)

(21) Appl. No.: 18/845,713

(22) PCT Filed: Mar. 10, 2023
(86) PCT No.: PCT/IB2023/000161

§ 371 Date: Sep. 10, 2024

(30) Foreign Application Priority Data
Mar. 11, 2022 (CH) CH000265/2022

(51) Int. Cl. A61F 5/00

(52) U.S. Cl. CPC A61F 5/0089; A61F 5/0076

(57) ABSTRACT

The invention is directed to a delivery device (10) for introducing and/or deploying an implant (12, 107) within the gastro-intestinal tract. The implant may be a bypass sleeve. The delivery device comprises a shaft assembly (24) with a longitudinal axis (L) and has an atraumatic tip component (26, 102) arranged at a distal end of the shaft assembly (24).

The present invention relates to the field of implants for insertion into the gastro-intestinal tract, and to delivery systems therefor. In some non-limiting aspects, the invention relates to a delivery system for a bypass sleeve for bypassing a portion of the bowel.

Various surgical techniques, and implants, have been proposed for treating obesity and diabetes. Surgical techniques include creation of gastric pockets and gastric bypasses of the stomach, duodenum and part of the jejunum. Implants such as bypass sleeves or liners have been proposed for insertion into the gastro-intestinal tract, to bypass the duodenum and optionally part of the jejunum.

Technical challenges remain in terms of delivering and deploying implants, for example bypass sleeves, within the gastro-intestinal tract, especially within the intestine. Systems introducible through a mouth and stomach have been proposed. However, such systems have limitations in terms of ease of use, and the distance that can be accessed within the intestine. For example, some clinicians currently regard the maximum accessible distance as being up to about 60 cm starting from the pylorus. The distance is limited by practical considerations such as delivery system size, friction against intestinal tissue, and ease of navigation without having to apply too high a pushing force to advance the delivery system. High forces may risk damaging intestinal tissue, and possibly perforating the intestinal wall.

The present invention has been devised to at least partly address and/or mitigate one or more of the above issues.

Broadly speaking, a first aspect of the invention provides a delivery system, also referred to as a delivery device, for introducing and deploying an implant, for example a bypass sleeve, within the gastro-intestinal tract, for example, extending within the duodenum and optionally at least partly within the jejunum.

The delivery system comprises a shaft assembly with a longitudinal axis. A atraumatic tip component is generally arranged at a distal end of the shaft assembly. The atraumatic tip may be carried by the distal end or it may be coupled to the distal end.

The shaft assembly may be formed by a pusher liner and/or an inner tube. The inner tube may be arranged within a pusher liner to form a pusher catheter.

The longitudinal axis of the delivery device may be understood as the direction along which the shaft assembly extends.

“Atraumatic” may typically refer to structures which are not damaging and/or do not deform the patient anatomy during the delivery procedure of the implant. For example, spherical shapes such as balls may have a reduced risk of perforating the bowels upon delivery of the intestinal sleeve.

The delivery system may be defined independently of, or in combination with, the implant, for example a bypass sleeve.

The delivery system may optionally comprise any one, or any combination of two of more, or all, of the following features:

(a) The delivery system may have an atraumatic tip component carried at a distal end of a shaft assembly having a longitudinal axis. The tip component may be at least partly rotatable about the axis, at least within a predetermined range of motion, for example, at least a full turn in either direction. The ability of the tip to rotate about the axis, at least somewhat, can assist in ease of navigating the tip component within the folded and tortuous passage of the intestine. Frictional contact may be reduced by the tip component being able to rotate as it comes into contact with tissue folds and bends.

(b) Additionally or alternatively, the delivery system may have an atraumatic tip component carried at a distal end of a shaft assembly, the tip component defining a clamp for clamping an end (e.g. downstream end) of a sleeve structure. The end of the sleeve structure may have a peripheral edge, for example, a circumferential edge. The clamp may be configured to clamp a first portion of the peripheral edge, leaving a second portion of the peripheral edge unclamped. The first portion may be a minor portion, and the second portion a major portion. By clamping only a portion of the peripheral edge of the sleeve structure, the amount of clamped material can be kept small, facilitating later release without compromising the clamping effect. The tip component can be made smaller than a similar device clamping all of the peripheral edge at the end of the sleeve structure. A small tip component can assist in ease of navigation through the intestine, and facilitate access deep into the intestine.

As used herein, the term “sleeve structure” covers a sleeve where the ends of the sleeve are at the end of the structure, and a sleeve that may be partly invaginated, such that at least one of the sleeve ends is not at the end of the structure. The end of the sleeve structure corresponds to the point at which the sleeve is folded inwardly on itself.

(c) Additionally or alternatively to (a) and/or (b) above, the delivery system may have an atraumatic tip component coupled to a distal end of a shaft assembly by a releasable connection, for example an anchor. The connection may optionally be released by manipulation of the shaft assembly by a handle or actuator remote from the distal end.

In some embodiments, the shaft assembly includes first and second elongate shaft components, for example, nested one within the other. The connection may be releasable by relative movement of one shaft component with respect to the other. Preferably, the relative movement is non-forward movement (e.g. non-distal movement). Non-forward movement may include, for example, retraction or rotation. By avoiding forward movement, there is considerably less risk of the exposed shaft assembly accidentally advancing into exposed tissue, and possibly damaging or piercing the tissue.

Whether or not a non-forward movement of the shaft assembly is used, various implementations of releasable connection are envisaged.

One example of releasable connection is an expandable and/or contractable gripper. The connection may be released by expanding and/or contracting the gripper. In one form, the gripper can change configuration between an expanded configuration and a contracted configuration. In one configuration (e.g. the expanded configuration), the gripper is configured to couple the shaft assembly to the atraumatic tip component, for example, by expanding within a socket of the tip component to grip the socket from within. In the other configuration (e.g. the contracted configuration), the gripper is configured to release the atraumatic tip, for example, by disengaging from within the socket of the tip component.

Another example of releasable connection may be a magnetic connection between first and second coupling parts. The magnetic connection may be formed by at least one permanent magnetic, optionally first and second permanent magnets. The connection may be releasable by manipulating the shaft assembly to force apart the first and second coupling parts. For example, one coupling part may be retracted into the shaft assembly to separate it from the opposite coupling part in the atraumatic tip. In another example, an electromagnetic coupling may be used. The coupling may be released by reducing or removing application of an electric current to an electromagnetic coupling part. The releasable connection may comprise a phase transition element having two distinct phases. For example, the phase transition element may comprise or consist of expandable elements of having an austenite and a martensite phase. The phase transition element may be adapted to provide a fixed connection between the shaft assembly and the atraumatic ball in a first phase (e.g. in the austenite phase), and release the atraumatic tip component from the shaft assembly in a second phase (e.g. in the martensite phase). The martensite phase in phase transition materials is generally more mechanically flexible than the austenite phase. Therefore, an expandable element may provide a fixed connection in austenite phase, wherein said connection may be released by being pulled out in the martensite phase in which it may be easily deformable.

In particular, the phase transition element may be formed by a shape memory alloy, e.g. Nitinol, wherein the first phase is a high-temperature phase and the second phase is a low-temperature phase. The shape memory alloy may be adapted such that a transition temperature, in particular a H-T transition temperature at which an austenite is transitioned to martensite, is usually below 4° C. for medical device applications designed for human body temperature environment.

(d) Additionally or alternatively to (a) and/or (b) and/or (c) above, an implant may comprise a bypass sleeve having an upstream end and a downstream end, the implant further comprising a downstream anchor coupled movably to the downstream end of the implant and/or bypass sleeve by one or more tethers.

The anchor may be relatively heavy, for example, having a weight of at least about 5 grams, optionally in a range of between about 5 grams and about 10 grams, so as to tend to be drawn by gravity to fall further within the intestine. The anchor can thus urge the downstream end of the sleeve in an antegrade direction with respect to the direction of flow of matter within the intestinal tract. During deployment, the anchor may assist in drawing the downstream end of the sleeve further downstream, to deploy the sleeve lengthwise further into the intestine. This may, for example, facilitate deployment of a long sleeve, for example, longer than 60 cm, optionally at least 70 cm, optionally at least 80 cm, optionally at least 90 cm, optionally at least 100 cm. After deployment, and whatever the length of the sleeve, the anchor can resist any tendency for the downstream end of the sleeve to move in a retrograde direction, for example, should the patient vomit. Vomiting is one condition in which conventional bypass sleeves may be subject to retrograde migration, risking blockage. Provision of an anchor as described herein may mitigate risk of such retrograde migration.

In some embodiments, prior to deployment of the sleeve, the downstream end of the sleeve may be partly invaginated into the main body of the sleeve, to define a sleeve structure with a shortened pre-deployment length. The length of the invaginated section may, for example, be about the same length as, or shorter than, the length(s) of the tether(s). In use, upon deployment, the anchor urges the downstream end in a distal (e.g. antegrade) direction, causing the invaginated sleeve to dis-invaginate, and revert to its fully extended length.

The implant may be inverted over the delivery system, in particular if the implant is attached to an anchor which is formed as an atraumatic tip of the delivery system. The atraumatic tip may be arranged at a distal end of the delivery system as described above.

The tether(s) attaching the anchor to the sleeve may be configured to be permanent, such that the anchor remains attached to the sleeve, and may be removed when the sleeve is extracted from the intestine after a certain period of use. Alternatively, the tethers may be biodegradable, such that the anchor will detach from the sleeve after a certain time, and pass naturally through the intestine to exit the body via the anus.

Optionally, the anchor is configured to form an atraumatic tip component of, or for, the delivery system. With such an arrangement, the anchor can serve a dual purpose of also having a function during introduction into the body prior to deployment of the sleeve. For example, the anchor may form a tip component having any of the features (a) and/or (b) and/or (c) described above.

A closely related second aspect of the invention provides a method of separating a tip component from a shaft assembly of a delivery system, the method comprising:
injecting fluid through the shaft assembly, to bear against the tip component, and separate from the shaft assembly.

Preferably, the fluid has a temperature adapted to bring a releasable connection into a low-temperature phase such as to release the atraumatic tip component from the shaft assembly. For example, the fluid may be a saline solution with a temperature below 4° C. Injecting the fluid may thus cool a phase transition element from body temperature (i.e. 37° C.) to below 4° C. and thus cause a phase transition, e.g. from austenite to martensite.

In some preferred embodiments, the system is assembled by introducing a pusher catheter with a snare into a sleeve-like implant. The snare is typically attached to an inner tube of the catheter, the inner tube being surrounded by a liner. A distal tip of the sleeve is fastened with the snare and pulled into an atraumatic ball. The distal portion of the delivery device is then arranged within the sleeve and held statically to one another.

Said system may be used to release the snare from a proximal side after reaching a designated location. Thus, the sleeve is detached from the delivery device and at least the inner tube and the snare may be retrieved from the treatment site via the pusher catheter liner. The atraumatic ball, which may have remained in place, may then be pushed in a distal direction by injection of pressurized saline so as to release the distal tip of the sleeve. When retrieving the pusher catheter liner, additional saline may be injected.

The method may further comprise releasing a coupling between the tip component and the shaft assembly prior to, and/or during, and/or after the step of injecting fluid.

Although certain aspects, features and advantages have been highlighted above, this is merely to aid understanding certain concepts used in the invention, without limiting the scope of protection. Protection is claimed for any novel idea or feature described herein and/or illustrated in the drawings, whether or not emphasis has been placed thereon.

Non-limiting embodiments are now described by way of example only, with reference to the accompanying drawings, in which:

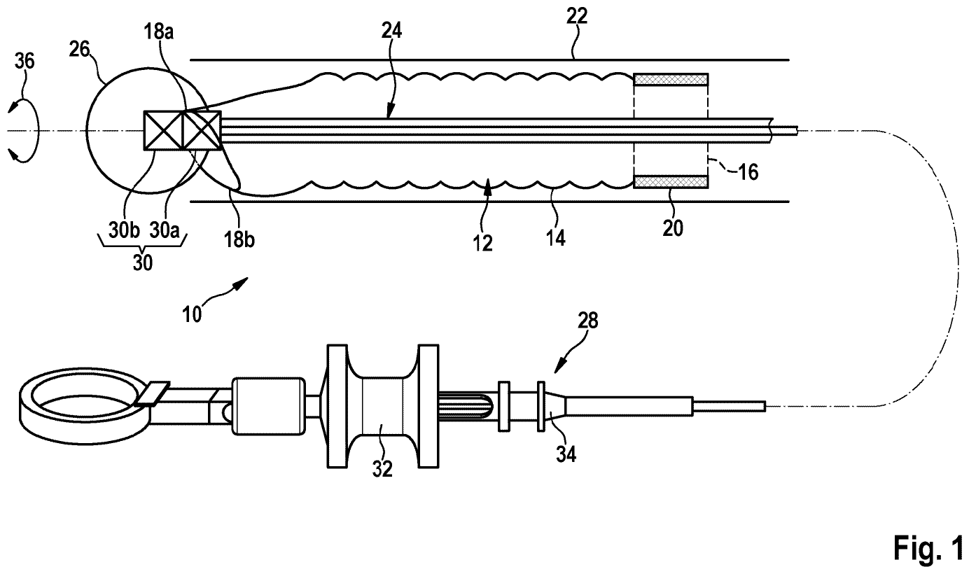

FIG. 1 is a schematic section illustrating a first embodiment of delivery system for a bypass sleeve;

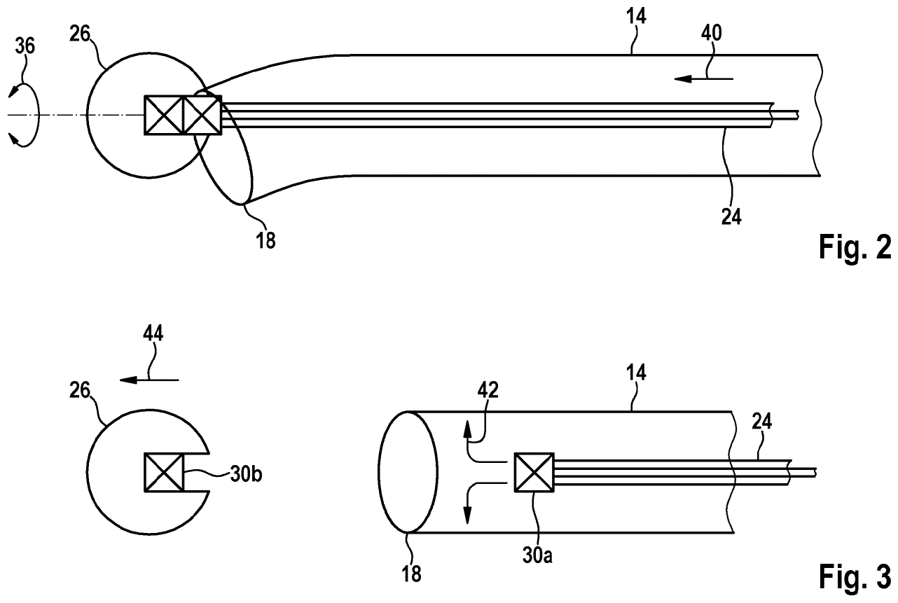

FIG. 2 is a schematic section illustrating the first embodiment in a partly deployed configuration;

FIG. 3 is a schematic section similar to FIG. 2 illustrating the embodiment in a fully deployed configuration.

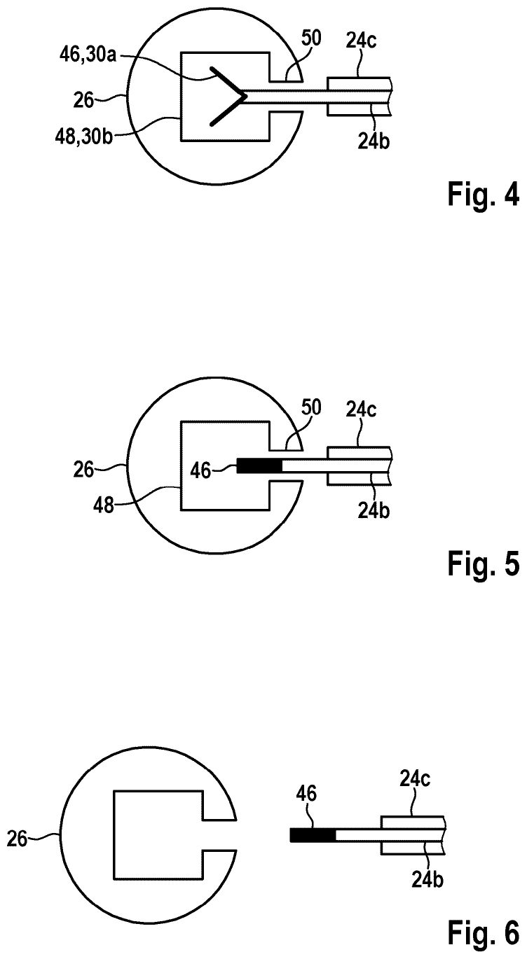

FIG. 4 is a schematic section illustrating a first example of tip structure of the first embodiment in a coupled configuration;

FIG. 5 is a schematic section similar to FIG. 4, illustrating the tip structure during release;

FIG. 6 is a schematic section illustrating the first example of tip structure in a released configuration;

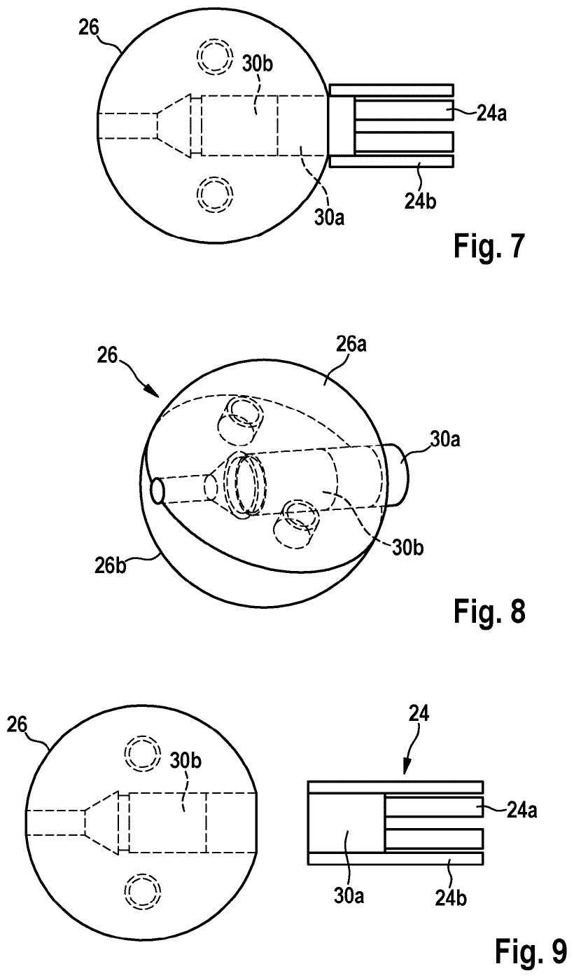

FIG. 7 is a schematic section illustrating a second example of tip structure of the first embodiment in a coupled configuration;

FIG. 8 is a schematic perspective section illustrating a construction of the tip structure;

FIG. 9 is a schematic section illustrating the second example in a released configuration;

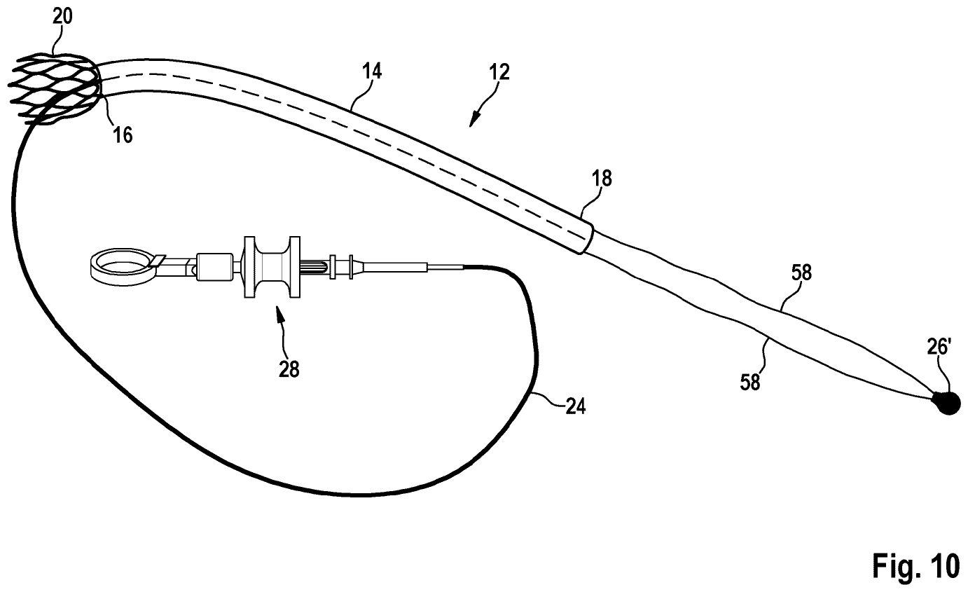

FIG. 10 is a schematic section illustrating a second embodiment of bypass sleeve device, and part of the delivery system;

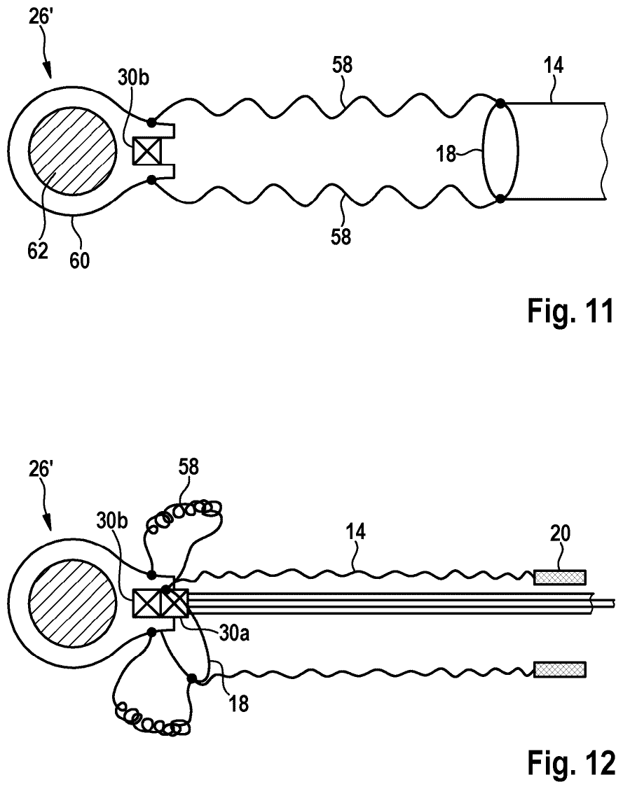

FIG. 11 is a schematic section illustrating a downstream end of the bypass sleeve device of FIG. 10;

FIG. 12 is a schematic section illustrating the sleeve device of the second embodiment in a pre-deployment configuration;

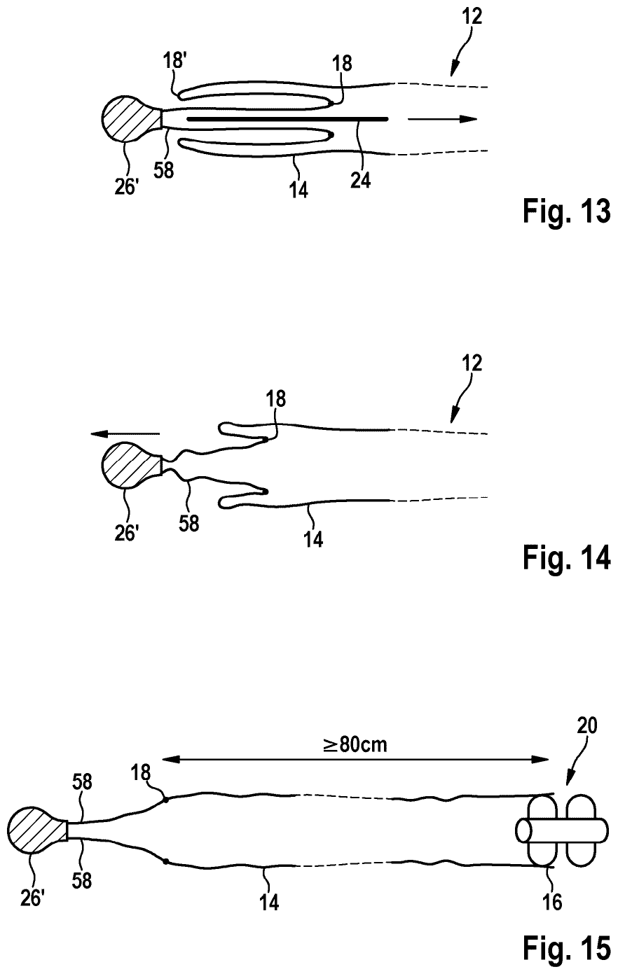

FIG. 13 is a schematic section illustrating a modification of the second embodiment in a pre-deployment configuration;

FIG. 14 is a schematic section illustrating the sleeve device of FIG. 13 transitioning to a partly deployed configuration; and

FIG. 15 is a schematic section illustrating the sleeve device of FIGS. 13 and 14 transitioning to a deployed configuration.

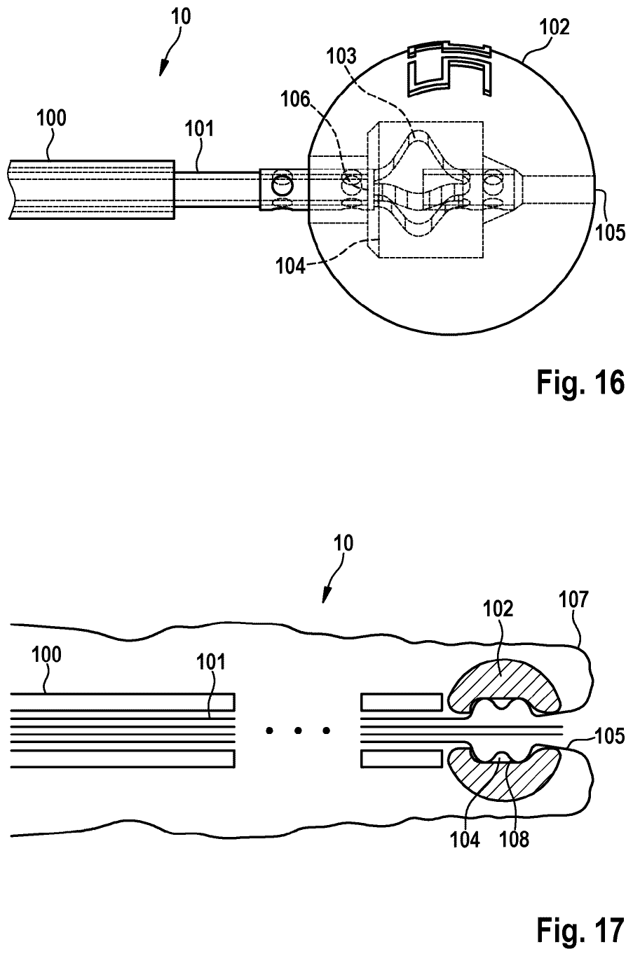

FIG. 16 is a schematic illustration of a section of a delivery device having a phase transition element forming a releasable connection.

FIG. 17 shows, schematically, the delivery device of FIG. 16 with a bypass sleeve.

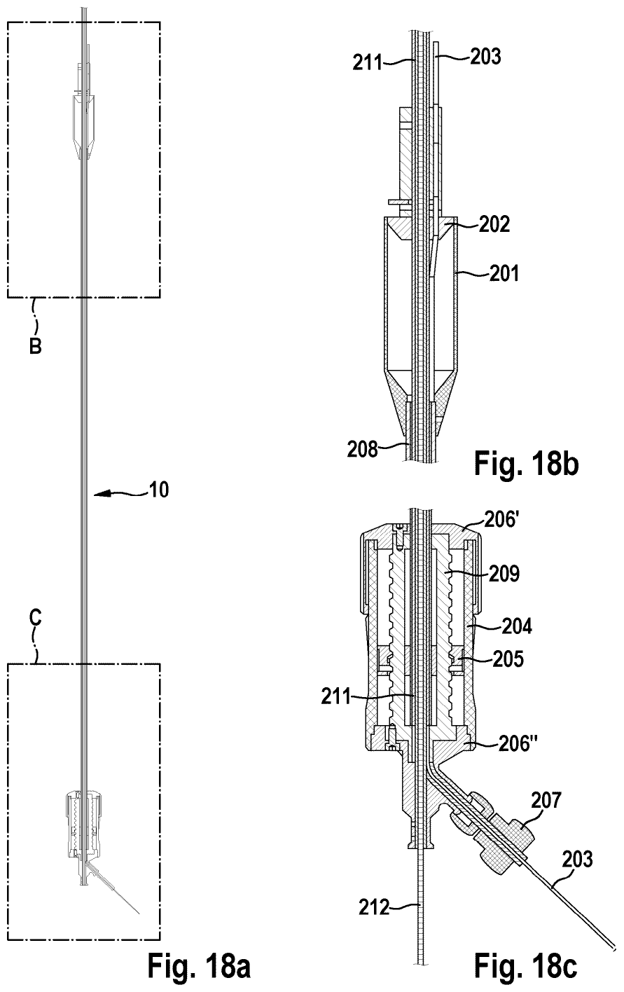

FIGS. 18a-18c show schematically a delivery device.

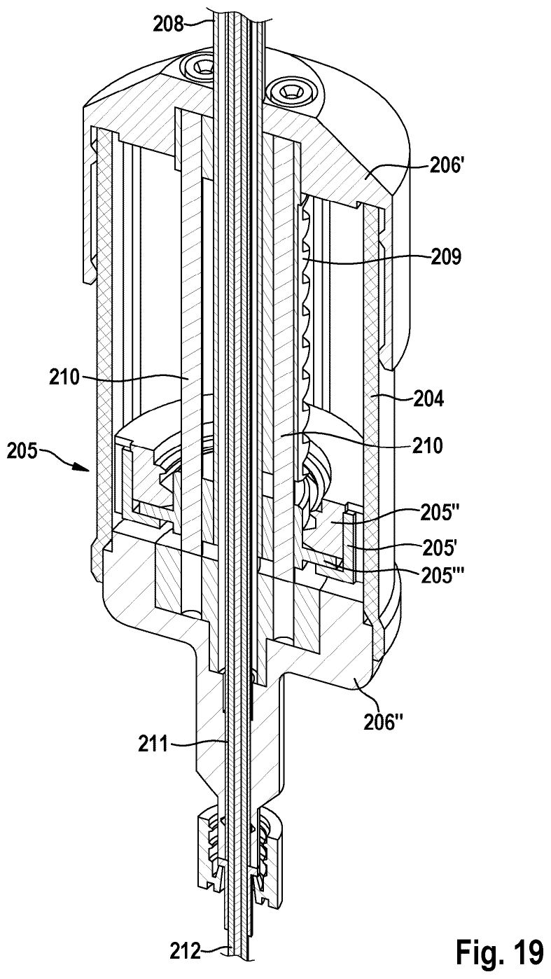

FIG. 19 shows a detailed view of the mechanism of the device of FIGS. 18a-c.

Referring to the drawings, the same reference numerals are used to denote the same or similar features whether or not described explicitly.

Referring to FIGS. 1 to 3, a delivery system 10 is illustrated for introducing and deploying an implant in the form of a bypass sleeve device 12 within the gastro-intestinal tract, for example, within the duodenum and optionally at least partly within the jejunum. The sleeve device 12 comprises a flexible tubular sleeve 14 having an upstream end 16 and a downstream end 18. The sleeve 14 may have a length (after deployment) of at least about 40 cm, optionally at least about 60 cm, optionally at least about 80 cm, optionally at least about 100 cm. The sleeve may have a diameter (after deployment) of between about 1.5 to 3 cm, preferably 2 cm and about 3 cm, optionally about 2.5 cm. One or more anchors 20 may be provided near or at the upstream end 16 for fitting at a pylorus of a patient. The anchors may, for example, include a self-expanding stent and/or an inflatable chamber.

The delivery system 10 comprises an introduction sheath 22 within which the sleeve device 12 is initially contained in a low-profile configuration prior to deployment. In the low-profile configuration, the sleeve device 12 is compressed axially, for example, to a length of not more than about 10 cm, optionally about 6 cm, and/or compressed radially. Particularly preferably, the sleeve may be foldable in an accordion-like structure. However, it is also possible to deliver the sleeve fully spread. For example, the sheath 22 may have a diameter (internal diameter and/or outer diameter) of between about 1 cm and 1.5 cm into which the sleeve device 12 is compressed radially. The sleeve 14 forms a sleeve structure in which the ends 16 and 18 of the sleeve also form the ends of the sleeve structure. These ends 16 and 18 of the sleeve and of the sleeve structure are referred to interchangeably.

The delivery system 10 further comprises a shaft assembly 24 passing through the sheath 22, and releasably coupled (or releasably coupleable) to a tip component 26. The tip component 26 may optionally be part of the delivery system 10, or part of the sleeve device 12 (as described later), or a separate component. A releasable coupling 30 comprises a first coupling part 30a carried by the shaft assembly 24, and a second coupling part 30b carried by the tip component 26. The shaft assembly 24 comprises at least first and second shafts 24a and 24b, movable one relative to another, by means of an actuator handle 28 at a proximal end of the system 10. The first shaft 24a may be nested within a tubular bore of the second shaft 24b, and a third shaft (not shown) may also optionally be provided. The handle 28 comprises a slider 32 movable axially with respect to a handle body 34, for manipulating the shaft assembly 24.

The tip component 26 has an atraumatic shape to facilitate sliding advancement of the delivery system 10 within the intestine. For example, the tip component 26 has an at least partly rounded distal surface. In the illustrated example, the tip component 26 is generally spheroid. As indicated by the arrow 36, the at least a part of the tip component 26 is able to rotate or twist, at least within a certain angle of rotation, about a longitudinal axis of the delivery system 10. For example, the coupling 30 may permit the entire tip component 26 to be rotated relative to the shaft assembly 24 and/or the sheath 22 and/or the sleeve device 12. In other embodiments, the tip component 26 could include an external shell that is rotatable about a non-rotatable inner part. The tip component 26 may be dimensioned to generally match the dimension of the sheath 22, to be able to plug the open end of the sheath 22. The tip component 26 may have a radial diameter (with respect to the longitudinal axis of the system) of between about 1 cm and 1.5 cm, optionally about 1.1 cm, or about 1.2 cm or about 1.3 cm or about 1.4 cm.

The tip component 26, and/or the coupling 30, is configured to clamp at least a portion of the peripheral edge of the downstream end 18 of the sleeve 24, for example, at the junction between the tip component 26 and the shaft assembly 24. In the illustrated example, only a first portion 18a of the peripheral edge is clamped, leaving a second portion 18b unclamped. The first portion 18a may be a minor portion (e.g. <50%) of the peripheral edge, and the second portion 18b may be a major portion (e.g. >50%).

In use, the delivery system 10 is advanced into the gastro-intestinal tract, for example, via a natural body opening such as a patient's mouth. The delivery system 10 can be advanced through the stomach through the pylorus and into the intestine, until the anchor 20 is aligned at a target position, for example, at the pylorus. Thereafter the sheath 22 is retracted with respect to the shaft assembly 24 to allow the sleeve 14 and the anchor 20 to expand (or be expanded) radially, such that the anchor 20 seats the upstream end 16 of the sleeve 14 at the target position.

Referring to FIG. 2, the shaft assembly 24 is then pushed (indicated by arrow 40), by means of the handle unit 28, to extend the sleeve 14 axially, by its downstream end 18, deeper into the intestine, for example through the duodenum and at least partly through the jejunum. The atraumatic shape of the tip component 24 and/or the ability to rotate about the longitudinal axis upon contact with the folds and bends of the intestinal tissue, facilitates navigation into the intestine.

Referring to FIG. 3, after having extended the sleeve 14, the tip component 26 is released from the downstream end 18 of the sleeve 18 by manipulation of the shaft assembly 24 using the handle unit 28. In the present embodiment, the coupling parts 30a and 30b are released by relative retraction of one shaft (e.g. 24a) with respect to another (e.g. 24b). For example, the slider 32 is retracted proximally relative to the handle body 34 to retract one shaft relative to the other.

If desired, a fluid (for example, saline, illustrated by arrows 42) may be injected through at least one shaft of the shaft assembly 24, to “blow” the tip component 26 free of the shaft assembly 24, and to release the clamping engagement of the downstream end 18 of the sleeve 14. The fluid may continue to be injected through the shaft assembly 24 as the shaft assembly is withdrawn proximally through the sleeve 14, to further expand the sleeve 14 radially outwardly into full deployment.

As indicated by arrow 44, the tip component 26 separates from the shaft assembly 24, and travels with fecal matter through the intestine to be discharged from the gastro-intestinal tract via the anus. The size and atraumatic shape of the tip component 26 facilitates natural discharge.

FIGS. 4 to 6 illustrate in more detail a first example of a tip structure and the coupling 30. The coupling part 30a of the shaft assembly 24 comprises an expandable and/or collapsible gripper, in the form of alligator jaws 46 mounted on an inner shaft of the shaft assembly 24. Axial advancement of the inner shaft relative to a surrounding tubular shaft 24b allows the jaws 46 to expand (e.g. self-expand) to an enlarged shape (FIG. 4). Axial retraction of the inner shaft collapses the jaws 46 into the surrounding shaft 24b (FIGS. 5 and 6). Other forms of expandable and/or collapsible coupling part 30a are also envisaged, but the alligator jaws 46 can provide a reliable mechanism that is small in size.

The coupling part 30b of the tip component 26 comprises an interior socket 48 within the tip component 26, optionally with a narrowed mouth 50. The socket 48 and/or mouth 50 is dimensioned to permit the coupling part 30a to slide into and/or out of the socket 48 when the coupling part 30a is in its collapsed configuration, but to lock the coupling part 30a inside the socket 48 when the coupling part 30a is in its expanded configuration. A third tubular shaft 24c may optionally be slidable over the second shaft 24c, to provide additional column strength to reinforce the second shaft 24b, and to provide a tight fit at the mouth 50 of the socket 48.

In use, unless the sleeve 14 is pre-attached, and prior to fitting the tip component 26, the alligator jaws 46 may be manipulated to grip or grasp the portion 18a of the downstream end of the sleeve 14 by collapsing the jaws 46. With the jaws 46 in the collapsed configuration, the jaws are introduced into the socket 48 of the tip component 26, to fit the tip component 26 and draw the gripped portion of the sleeve into the tip component 26. Thereafter, the jaws 46 are manipulated to expand within the socket 48, thereby locking the tip component 26 to the shaft assembly 24 and clamping the portion 18a of the sleeve 14 at the junction between the tip component 26 and the shaft assembly 24. The third shaft 24c may be advanced into the mouth 50 of the socket 48 to form a tight fit, that helps to further clamp the sleeve 14 at the mouth 50. Such an arrangement clamps the sleeve 14 while also allowing the tip component 26 to turn about the axis of the shaft assembly 24.

Referring to FIGS. 5 and 6, when it is desired to release the coupling 30, the alligator jaws 46 are retracted into the shaft 24b. The third shaft 24c may also be retracted out of the mouth 50 of the socket 50. The tip component 26 is free to separate from the shaft assembly 24, and to release the clamped portion of the sleeve 14 (not shown in FIG. 6).

FIGS. 7 to 9 illustrate a second example of tip structure and coupling 30, using magnetic attraction. The first and second coupling parts 30a and 30b comprise magnetically attractable elements. For example, both parts 30a and 30b may be implemented as magnets arranged in mutually attracting orientations. Alternatively, one part 30a or 30b may be a magnet, and the other part may 30b or 30a, respectively, may be a ferromagnetic element.

The first coupling part 30a is secured to the distal end of a first shaft 24a of the shaft assembly 24. The first shaft 24a is advanceable and retractable relative to a surrounding second shaft 24 under control of the handle unit 28. The second coupling part 30b is secured within an internal socket recess of the tip component 26. Referring to FIG. 8, the tip component 26 may, in some examples, be made in two halves 26a and 26b that, when assembled together, secure the second coupling part 30b within the tip component 26.

In use, unless the sleeve 14 is pre-attached, and prior to fitting the tip component 26, the portion 18a of the downstream end of the sleeve 14 to be clamped, is arranged to cover the first coupling part 30a. Thereafter, the tip component 26 is fitted to the shaft assembly, such that the portion 18a of the sleeve 14 is clamped between the magnetically attracting parts 30a and 30b. Such an arrangement clamps the sleeve 14 while also allowing the tip component 26 to turn about the axis of the shaft assembly 24.

Referring to FIG. 9, when it is desired to release the coupling 30, the first shaft 24a is retracted with respect to the second shaft 24b. The counter force to the magnetic attraction is applied through the second shaft 24b, thereby pulling apart of the first and second coupling parts 30a and 30b and, at the same time, releasing the sleeve 14 from clamping engagement between the coupling parts 30a and 30b. The tip component 26 is free to separate from the shaft assembly 24 and release the downstream end 18 of the sleeve 14 (not shown in FIG. 9).

FIGS. 10 to 12 illustrate a second embodiment in the form of an apparatus comprising a sleeve device 12 including a sleeve 14 and a downstream anchor 26′ attached to the sleeve 14 by means of one or more tethers 58. The downstream anchor 26′ is relatively heavy, to promote the anchor falling within the intestine, and being drawn by intestinal motion. For example, the downstream anchor 26′ may have a weight between about 1 gram and about 10 grams, optionally between about 5 grams and about 10 grams, optionally about 5 grams. The anchor 26′ can thus urge the downstream end 18 of the sleeve 14 in an antegrade direction with respect to the direction of flow of matter within the intestinal tract. During deployment, the anchor 26′ may assist in drawing the downstream end 18 of the sleeve 14 further downstream, to deploy the sleeve lengthwise further into the intestine. This may, for example, facilitate deployment of a long sleeve, for example, longer than 60 cm, optionally at least 70 cm, optionally at least 80 cm, optionally at least 90 cm, optionally at least 100 cm. After deployment, and whatever the length of the sleeve 14, the downstream anchor 26′ can resist any tendency for the downstream end 18 of the sleeve 14 to move in a retrograde direction, for example, should the patient vomit.

The tethers 50 may have a length of between about 10 cm and about 50 cm, to allow the downstream anchor 26′ to distance itself from the downstream end 18 of the sleeve 14. Such a distance avoids the downstream anchor 26′ from obstructing the exit of the sleeve 14 in use, and also provides a degree of decoupling between the anchor 26′ and the sleeve 12 within the intestine while still achieving a reliable anchoring effect.

The anchor 26′ is optionally made as a moulded plastics shell surrounding a heavy, e.g metallic, mass optionally shaped as a ball. The anchor 26′ may be elongated (e.g. at least slightly longer than wide). For example, the downstream anchor 26′ may have a length of about 2 cm, and a diameter of between about 1 cm and about 1.5 cm, optionally about 1.2 cm. In this example, the anchor 26′ has an at least partly rounded shape, embodied as a pear-shaped or teardrop profile.

The tethers 58 may be configured to be permanent, such that the anchor 26′ remains attached to the sleeve 14, and may be removed with the sleeve 14 when the sleeve 14 is extracted from the intestine after a certain period of use. Alternatively, the tethers 58 may be biodegradable, such that the downstream anchor 26′ will detach from the sleeve 14 after a certain time, and pass naturally through the intestine to exit the body via the anus.

Referring to FIGS. 11 and 12, in addition to functioning as a downstream anchor, the anchor 26′ may also function as a tip component for a delivery system 10, similar to the first embodiment. The delivery system comprises a shaft assembly 24 carrying a coupling part 30a for releasable coupling to a complementary coupling part 30b carried by the downstream anchor 26′. The coupling parts 30a and 30b may form a mechanical coupling as in the first example or a magnetic coupling as in the second example.

When the sleeve device 12 is installed in the delivery system 10, the tethers 58 collapse axially, allowing the downstream anchor 26′ to approach the downstream end 18 of the sleeve 14 for clamping a portion 18a of the downstream sleeve end 18 at the junction between the downstream anchor 26′ and the shaft assembly 24 (FIG. 12). The tethers 58 may limit to some extent the ability of the downstream anchor 26′ to have unlimited rotation, but the tethers provide sufficient slack material that rotation of the downstream anchor 26′ is not limited to any practical extent.

By configuring the downstream anchor 26′ also to act as a tip component clamping the sleeve 14 and attached to the shaft assembly 24, the delivery system 10 can be used in the same way as described above, to deploy and extend the sleeve 14 within the intestine. When the coupling 30 is released, the downstream anchor 26′ separates from the shaft assembly 24, and is free to be pulled further into the intestine by its weight and/or by flow of fecal matter, and/or by natural peristaltic motion of the bowel wall tissue.

FIGS. 13-15 illustrate a modification of the second embodiment, allowing an even longer sleeve 14 to be deployed. For example, the sleeve may have a length of at least 80 cm, optionally about or at least about 100 cm, or more. In this example, the downstream end 18 of the sleeve 14 is at least partly invaginated into the remainder of the body of the sleeve 14, to define a sleeve structure with an upstream end corresponding to the upstream end 16 of the sleeve, and a downstream end 18′ corresponding to the position at which the sleeve 14 folds on itself to form the invaginated shape. The length of the invaginated portion may be about the same length as the tethers 58, or shorter. The tethers 58 accommodate the downstream end 18 of the sleeve being pushed internally.

The downstream anchor 26′, functioning as a tip component for the delivery system clamps the downstream end 18′ of the sleeve structure, in a similar manner to that described above. Referring to FIGS. 14 and 15, when the coupling is released, the downstream anchor 26′ moves in the antegrade direction, to pull out the invaginated portion of the sleeve 14, such that the sleeve 14 can extend to its full length (FIG. 15).

FIG. 16 shows schematically a section of a delivery device 10. The delivery device 10 comprises a pusher catheter formed by a pusher liner 100 and an inner tube 101 arranged within the pusher liner 100. At a distal end of the pusher catheter, an atraumatic ball 102 is arranged. The atraumatic ball 102 is coupled to the pusher catheter 100, 101 by a releasable connection, which is here formed by radially expanded bands 103. Here, three bands 103 are present, but it will be understood that more or less bands would work as well. The bands 103 are made of a nitinol alloy with a phase transition temperature (Af temperature Austenite-finish temperature) of approximately 20° C. Therefore, at room temperature or above (e.g. at body temperature), the bands 103 are mechanically stiff and secure the pusher catheter 100, 101 with respect to the atraumatic ball 102 by being locked in an inner cavity 104 of the atraumatic ball 102. The cavity 104 has a diameter which is larger than a proximal opening 106 through which the pusher catheter 100, 101 extends. The bands 103 are generally sized that they fit in the cavity 104 without necessarily exerting a force on the cavity walls, but such the bands 103 cannot fit through the proximal opening without deformation. Generally, in the austenite phase, the bands 103 are therefore prevented from being removed from the cavity 104 because of the rigidity of the bands 103 in the austenite phase. If the bands 103 are cooled down below the phase transition temperature of 4° C., the bands 103 are transitioned into the martensite phase. In the martensite phase, the bands can be deformed with a comparably low force. Accordingly, they can be pulled out of cavity 104, through opening 106, without damaging the atraumatic ball or requiring excessive force. Cooling may be achieved by injection of a cooled saline. The atraumatic ball 104 further has a distal opening 105 for attachment of an implant, such as a bypass sleeve (not shown, see FIG. 17).

FIG. 17 schematically shows the delivery device 10 of FIG. 16. Similar elements as in FIG. 16 are not described again for clarity. Here, an implant 107 in the form of a bypass sleeve is attached to the atraumatic ball 102. To this end, a distal part of the implant 107 is held by a snare 108 which is arranged within the distal opening 105 of the ball 102. The implant 107 is inverted over the delivery device 10.

The inner catheter 101 comprises the snare 108 and holds the sleeve tip. As such, the sleeve 107 may prevent the ball 102 from slipping out when the outer catheter 100 pushes the ball 102 forward within the bowels. The mechanism therefore provides attachment in that the ball 102 is held by the folded sleeve 107 on one end and by the tip of the outer catheter 100 on the other end.

In some embodiments, the sleeve 107 may be pinched against an inner wall of the atraumatic ball by the pusher liner 101. This may provide additional securing in combination with the snare 108, or may replace the snare 108 entirely. In addition, the sleeve 107 may have a punched hole in vicinity of its distal end which may also be used for securing to the atraumatic ball 102. To this end, in some embodiments, the sleeve 107 may be pinched at a proximal position by the catheter 101, and distally by the ball 102. The ball 102 may be held internally by nitinol anchor as described herein. Release of the sleeve 107 may be achieved by pulling the nitinol anchor from the ball cavity, as described herein, such as to release the ball 102 and free up the sleeve 107.

In such embodiments, once sleeve deployment in a patient's intestine is competed, ice-cold saline may be injected from a proximal tip of the pusher tube 101, which reduces the temperature and consequently the rigidity of bands 103. Therefore, the pusher tube 101 and the bands 103 attached thereto may be pulled through the pusher liner 100.

After pusher tube 101 and bands 103 are fully retrieved, hot saline may be injected through pusher liner 100 to push the atraumatic ball 102 away from the sleeve 107, in a distal direction. The atraumatic ball 102 may then pass through the intestine and leave the body through natural bowel movements. The pusher liner 100 may be retrieved gradually while additional saline is injected in order to inflate the sleeve 107 and assuring its patency.

FIG. 18a shows an overview of a delivery device 10 which will be described in more detail in FIGS. 18b and 18c.

FIG. 18b shows a detailed view of panel B of FIG. 18a, showing a distal portion of a delivery device 10 comprising a capsule 201 for gastric stent (not shown) including a stent holder 202. A sleeve pusher catheter 212, which may include a pusher tube and/or a pusher liner as described above, extends through the capsule along with an inflation tube 203. It will be understood that a distal position of the capsule 201, a distal portion as shown in FIGS. 16 and 17 may be arranged, but is not shown here. Accordingly, the delivery device may be used to deliver a sleeve-like implant and e.g. a gastric stent connected thereto. Such an implant may be released by pulling of the distal part 201. For example, the distal part 201 may be connected to an outer shaft 208 which is configured to pull the distal part 201 back (see FIG. 18c).

FIG. 18c shows a detailed view of panel C of FIG. 18a, showing a handle portion for controlling part of the delivery device 10. An inner shaft 211 extends through the handle and is fixedly connected to the handle. The outer shaft (see FIG. 19, 208) is arranged concentrically around the inner shaft 211 and is slideable with respect to the inner shaft 211 and the handle. The sleeve pusher catheter 212 extends inside the inner shaft 211. Separately, the inflation tube 203 extends therethrough and opens in a separately arranged inflation port 207. The handle comprises a static nuts 206′, 206″ and a releasing nut 204 and further includes a sliding mechanism 205 which will be described in FIG. 19 below. Rotation of the releasing nut 204 with respect to the static nuts 206′, 206″ causes the sliding mechanism 205 to slide along a longitudinal direction, thereby moving the outer shaft 208 in the same direction. As a result, the outer shaft 208 is moved in a longitudinal direction.

FIG. 19 shows the functioning principle of the element of FIG. 18c in more detail. The static nuts 206′, 206″ are fixedly connected to the delivery device. The releasing nut 204 is rotatable about the longitudinal axis of the delivery device with respect to the static nuts 206′, 206″. The sliding mechanism 205 is comprises an inner slider 205′ and an outer slider 205″ which together form a rotatable portion of the sliding mechanism. The rotatable portion is fixedly attached to the releasing nut 204 in such a way that it rotates when the releasing nut 204 is rotated. However, the rotatable portion is arranged such as to freely slide with respect to the releasing nut in a longitudinal direction. For example, longitudinal grooves with corresponding protrusions may be used to transmit a rotational movement from the releasing nut 204 to the inner and outer sliders 205′, 205″. The inner slider 205′ has an inner thread which is in operable connection with an outer thread of an inner screw 209. Therefore, when the releasing nut 204 is rotated, the rotatable portion of the sliding mechanism 205 is rotated as well and moves along the inner screw 209 and therefore moves longitudinally with respect to the releasing nut 204. The sliding mechanism 205 further comprises a fixed slider 205′″ which is held by a groove formed by the inner and outer sliders 205′, 205″. Therefore, a rotational movement from the rotatable portion of the sliding mechanism 205 is not transmitted to the fixed slider 205′″. Instead, the fixed slider 205′″ is held, in a rotationally fixed position, in the inner screw 209. The inner screw 209 has a slit through which the fixed slider 205′″ extends and connects to the inner and outer slider 205′, 205″. However, the groove which holds the fixed slider 205′″ transmits the translational movement of the rotatable portion of the sliding mechanism 205. The fixed slider 205′″ is fixedly attached to the outer shaft 208. Therefore, when the releasing nut 204 is rotated, the outer shaft 208 is moved along the longitudinal axis, but the outer shaft 208 is not rotated. Two inner bars 210 are arranged inside the inner screw to secure the inner screw to the static nuts 206′, 206″. As described in the context of FIGS. 18a-18c, the translational movement of the outer shaft 208 may, for example, be used to pull back a distal part 201 such as to release a gastric stent, for example. A sleeve pusher catheter (see FIG. 17, for example) may be arranged in the outer shaft 208 in some embodiments.

It will be understood that the handle shown in FIG. 18c may be used, additionally or alternatively, to control other parts of the delivery device 10. The delivery device shown in FIGS. 18a-19 may be combined with any embodiment described herein.

It is emphasized that the foregoing description is merely illustrative of example forms of the invention, and that many modifications and equivalents can be used without departing from the scope and/or principles of the invention.

1-20. (Canceled)

21. A delivery device for introducing and/or deploying an implant within the gastro-intestinal tract, comprising a shaft assembly with a longitudinal axis, and having an atraumatic tip component arranged at a distal end of the shaft assembly.

22. The delivery device according to claim 21, wherein the atraumatic tip component is carried at the distal end, wherein the atraumatic tip component is at least partly rotatable about the longitudinal axis.

23. The delivery device according to claim 21, wherein the atraumatic tip component is carried at the distal end, further wherein the atraumatic tip component defines a clamp for clamping an end of a sleeve structure.

24. The delivery device according to claim 21, wherein the atraumatic tip component is coupled to the distal end by a releasable connection.

25. The delivery device according to claim 21, wherein the shaft assembly includes first and second elongate shaft components.

26. The delivery device according to claim 24, wherein the releasable connection is releasable by relative movement of the first or second shaft component with respect to the respective other shaft component.

27. The delivery device according to claim 26, wherein the releasable connection is formed by at least one of an expandable gripper and contractable gripper.

28. The delivery device according to claim 26, wherein the releasable connection is formed by a magnetic connection between first and second coupling parts.

29. The delivery device according to claim 26, wherein the releasable connection comprises a phase transition element having two distinct phases, said phase transition element being adapted to provide a fixed connection between the shaft assembly and the atraumatic tip in a first phase, and release the atraumatic tip component from the shaft assembly in a second phase.

30. The delivery device according to claim 29, wherein the phase transition element is formed by a shape memory alloy, wherein the first phase is a low-temperature phase, and the second phase is a high-temperature phase.

31. A system comprising a delivery device and an implant, the implant having an upstream end and a downstream end, the implant further comprising a downstream anchor coupled movably to the downstream end of the implant by at least one tether.

32. The system according to claim 31, wherein the implant comprises a bypass sleeve, the bypass sleeve having a length of at least 60 cm.

33. The system according to claim 31, wherein the anchor has a weight of at least 5 g.

34. The system according to claim 32, wherein the at least one tether is configured to be permanently attached to the sleeve.

35. The system according to claim 31, wherein the at least one tether is biodegradable.

36. The system according to claim 32, wherein the downstream end of the sleeve is at least partly invaginated into the main body of the sleeve such as to define a sleeve structure with a shortened pre-deployment length.

37. The system according to claim 31, wherein the anchor is formed by an atraumatic tip component of the delivery device.

38. The system according to claim 31, wherein the implant is attached to the anchor and inverted over the delivery device.

39. A method of separating a tip component from a shaft assembly of a delivery system, the method comprising injecting fluid through the shaft assembly, to bear against the tip component, and separate from the shaft assembly.

40. The method of claim 39, wherein the fluid has a temperature adapted to bring a releasable connection into a low-temperature phase such as to release the tip component from the shaft assembly.
